(19) United States
(12) Patent Application Publication
(10) Pub. No.: US 2002/0127654 A1
(43) Pub. Date: Sep. 12, 2002

(54) Compositions and methods for production cell culture

(76) Inventors: Virginia L. Price, Seattle, WA (US); Sharon T. Wong-Madden, Seattle, WA (US)

Correspondence Address: IMMUNEX CORPORATION, LAW DEPARTMENT, 51 UNIVERSITY STREET, SEATTLE, WA, 98101

(21) Appl. No.: 10/080,428

(22) Filed: Feb. 22, 2002

Related U.S. Application Data
(60) Provisional application No. 60/270,943, filed on Feb. 22, 2001.

(51) Int. Cl. C12P 21/02; C12N 5/06

(52) U.S. Cl. 435/69.5; 435/325; 435/320.1

(57) ABSTRACT

The invention provides improved methods of recombinant protein production in cell culture. More specifically, the invention relates to the activation of NF-kappa-B transcription factor complex in cells so as to improve production characteristics.

### RELATED APPLICATION DATA

This application claims the benefit of provisional U.S. application No. 60/270,943, filed Feb. 22, 2001, the entire disclosure of which is incorporated by reference herein.

### FIELD OF THE INVENTION

The invention is in the field of eukaryotic cell culture, and improved methods of recombinant protein production. More specifically, the invention relates to the activation of the NF-kappa-B signaling pathway in cultured eukaryotic cells so as to obtain cell cultures with advantageous properties.

### BACKGROUND OF THE INVENTION

The NF-kappa-B transcription factor complex is a pleiotropic activator that participates in the induction of a wide variety of cellular and viral genes. The active complex is composed of two subunits of the Rel/NF-kappa-B family. The members of this family include p65 (also known as RelA, NFKB3 and NF-kappa-B p65 subunit) p50, cRel, p52 and RelB. The genes for these subunits have been cloned and the N-termini show considerable homology to the product of the oncogene rel.

NF-kappa-B normally exists in the cytoplasm as a homodimer or heterodimer that is bound to a family of inhibitor proteins known as IKB. IKB-alpha (Inhibitor-Kappa-B-alpha) and IKB-beta. The IKB family consists of IKB-alpha, IKB-beta, IKB-gamma, and other IKB related proteins, such as Bc1-3. Phosphorylation of the IKB's triggers them to be ubiquitinated and then degraded, thereby releasing NF-kappa-B, which is then free to translocate to the nucleus and activate expression of its downstream targets.

Two closely related kinases responsible for IKB phosphorylation (and hence NF-kappa-B activation) have been identified and are termed Inhibitor-Kappa B Kinase-alpha (IKK-1, for I-Kappa-B Kinase 1) and Inhibitor-Kappa B Kinase-beta (IKK-2, for I-Kappa-B Kinase 2) (DiDonato et al., Nature, 1997, 388, 548-554; Zandi et al., Science, 1998, 281, 1360-1363; Zandi et al., Cell, 1997, 91, 243-252). In these studies, Inhibitor-Kappa B Kinase-alpha and Inhibitor-Kappa B Kinase-beta were found to directly phosphorylate IKB-alpha and IKB-beta.

NF-kappa-B proteins have been shown to be involved in a diverse set of signaling pathways involving stress, cancer, growth, infection, and inflammation. NF-kappa-B has also been implicated in apoptotic signaling, protecting cells from programmed cell death under some circumstances and accelerating apoptosis in others.

### SUMMARY OF THE INVENTION

The invention is based, in part, on the discovery that activation of the NF-kappa-B transcription factor complex leads to increased expression of a recombinant gene of interest. Accordingly, in one aspect, the invention provides an eukaryotic host cell genetically engineered to activate the NF-kappa-B transcription factor complex, and to express a protein of interest as an extracellular product. In particular embodiments, the cells are genetically engineered to express a NF-kappa-B transcription factor. Preferred NF-kappa-B transcription factors are p65, p50, cRel, p52 and RelB. The protein of interest can be any recombinant protein of economic interest. Examples of such proteins include but are not limited to a soluble TNF receptor, a soluble IL4 receptor, a soluble IL-1 type II receptor, a soluble Flt3 ligand, a soluble CD40 ligand, an erythropoeitin, an antibody, and hormones, to name just a few. Optionally, the gene for the protein of interest and/or the NF-kappa-B gene(s) can be linked to a selectable marker. Preferred host cells are mammalian cells, and more preferably mammalian cells that are grown in culture. In addition, the host cell can be adapted to grow in serum-free and/or protein-free and/or peptone-free medium.

In another related aspect, the invention provides a method of producing a protein of interest, the method comprising culturing an eukaryotic host cell genetically engineered to activate the NF-kappa-B transcription factor complex, and to express a protein of interest as an extracellular product, under conditions such that the protein of interest is expressed and secreted. Optionally, the method entails collecting and/or purifying the protein of interest from the cell culture.

In still other aspects, the invention relates to a method of producing a cell for production of a protein of interest, the method comprising genetically engineering a cell to express a gene that encodes a protein of interest, and to activate the NF-kappa-B transcription factor complex. The cells can be genetically engineered in any order or simultaneously. In addition, the cells can be transiently transformed or stably transformed.

### BRIEF DESCRIPTION OF THE FIGURE

FIG. 1 illustrates the effect of over expressing a p65 NF-kappa-B subunit on expression of a recombinant CD40Fc fusion protein. Cell lines (COS, CV-1, and 293 MSR) were transiently co-transfected as indicated. Seven days post-transfection, production of CD40Fc was assayed as a function of culture volume.

### DETAILED DESCRIPTION OF THE INVENTION

Overexpression of NF-kappa-B can be used to increase the expression of recombinant genes of interest. While not intending to be limiting, it is believed that the invention finds particular use in expressing coding sequence for genes of interest that are expressed under the control of promoters bearing NF-kappa-B binding sites. Such promoters include but are not limited to viral promoters commonly used to overexpress recombinant proteins (e.g., the CMV promoter and tar have three and two NF-kappa-B binding sites, respectively). The invention can be used for both transiently- and stably-transfected cells.

In addition, over-expression of NF-kappa-B can result in a more viable culture due to less apoptosis. Over-expression of NF-kappa-B can render the host cells growth-factor independent through increased transcription of endogenous growth factor genes.

Accordingly, cells with advantageous properties are created by genetically engineering cells to activate the NF-kappa-B transcription factor complex. Activation of the NF-kappa-B transcription factor complex can be accomplished by increasing the levels of active NF-kappa-B gene products, or by reducing the levels of the cellular inhibitors of the active NF-kappa-B transcription factor complex.

For purposes of the invention, an “NF-kappa-B gene” is a member of the conserved family of genes whose subunits form homodimeric and heterodimeric proteins that bind to NF-kappa-B binding sites and positively regulate genes. Generally, NF-kappa-B genes show homology to the rel oncogene. Preferably, the NF-kappa-B genes encode a protein with a region that is at least 50% homologous, more preferably 60% homologous, even more preferably 70% homologous, yet still more preferably 80% homologous, even more preferably 90% homologous, to the 300 amino acid rel region in one of the identified mammalian NF-kappa-B genes p65, p50, cRel, p52 and RelB (see Ghosh et al., 1998, Annu. Rev. Immunol. 16:225-260). Homology and identity can be calculated using the BLAST program version BLASTP 2.2.2, with the following parameters: matrix Blosum62, gap open 11, gap extension 1, x_dropoff 50, expect 10 and word size 3, filter on.

The products of the NF-kappa-B genes are NF-kappa-B transcription factors. NF-kappa-B genes and their products that are advantageously upregulated in the compositions and methods of the invention include but are not limited to p65, p50, cRel, p52 and RelB, and variants that are at least 80%, and/or at least 90%, homologous to these transcription factors.

As shown by the experimental data reported herein, either an empty expression vector, or an expression vector that encodes a caspase-resistant mutant of the p65 subunit of the NF-kappa-B, was co-transfected along with a vector that codes for the expression of a gene of interest. Transient transfections were performed on several different cell lines. Production levels of the gene of interest were greatly increased in the presence of the NF-kappa-B containing expression vector. The complete sequence for p65 can be found in Genbank Accession No. M62399. The caspase-resistant p65/NF-kappa-B subunit is described in Levkau et al., 1999, Nat. Cell. Biol. 1(4):227-33). Protease resisting versions of NF-kappa-B subunits are advantageous because more sustained levels of active p65/NF-kappa-B subunit can be maintained. The cells can also be genetically engineer to overexpress two or more of the NF-kappa-B genes.

Another way of activating NF-kappa-B that is within the scope of the invention is to underexpress or knock out expression of one or more of the family of inhibitor proteins known as IKB. The IKB family consists of IKB-alpha, IKB-beta, IKB-gamma, and other IKB related proteins, such as Bc1-3.

Still another way of activating NF-kappa-B that is within the scope of the invention is to increase the expression or activity of the IKK family members which, when activated, target for degradation the IKB family members. As noted above, IKK family members include but are not limited to IKK-1 and IKK-2. Also encompassed within the invention is the activation of the NF-kappa-B transcription factor complex by any combination of the above.

By the term “genetically engineered” is meant any recombinant DNA or RNA method used to create a eukaryotic host cell that expresses a gene at elevated levels, at lowered levels, or a mutant form of the gene. In other words, the cell has been transfected, transformed or transduced with a recombinant polynucleotide molecule, and thereby altered so as to cause the cell to alter expression of a desired protein. Methods and vectors for genetically engineering cells and/or cell lines to express a protein of interest are well known to those of skill in the art; for example, various techniques are illustrated in Current Protocols in Molecular Biology, Ausubel et al., eds. (Wiley & Sons, New York, 1988, and quarterly updates) and Sambrook et al., Molecular Cloning: A Laboratory Manual (Cold Spring Laboratory Press, 1989). Genetic engineering techniques include but are not limited to expression vectors, targeted homologous recombination and gene activation (see, for example, U.S. Pat. No. 5,272,071 to Chappel) and trans activation by engineered transcription factors (see, for example, Segal et al., 1999, Proc. Natl. Acad. Sci. USA 96(6):2758-63).

Methods of upregulating a NF-kappa-B gene product include overexpression of the encoded wild-type protein, expression of an altered protein (e.g., partly or constitutively activated mutant, or a protease resistant form as described below), or a genetically engineering the cells to express a protein with an altered cellular distribution (e.g., nucleoplasm) that has increased activity. Although overexpression of the NF-kappa-B gene product is desired, it should be noted that expression of extremely high levels of any gene product, especially NF-kappa-B gene products, is detrimental or even lethal to a cell, and as such should be avoided. Titration of the appropriate expression level can be manipulated in any of a number of ways (e.g., by choice of promoter or change in gene copy number) and is within the skill of those in the art.

Preferably, the cells are genetically engineered to express a NF-kappa-B gene product or an IKK that is homologous to, or derived from the same species, as that of the cell. However, as NF-kappa-B genes tend to be well conserved, it is expected that even expression of heterologous gene products will be advantageous. For example, in the non-limiting embodiments described below, the human p65 subunit was expressed in simian and murine cells, and successfully increased expression levels of a gene of interest.

Methods of downregulating the NF-kappa-B inhibitors include the use of ribozyme technologies, antisense and triple helix technologies, targeted homologous recombination to knockout or otherwise alter the endogenous gene, and expression of dominant negative mutant forms of the NF-kappa-B inhibitor gene product. Such methods of upregulating and/or downregulating the expression of gene products are well known to those of skill in the art.

By a “heterologous regulatory element” is meant a genetically encoded element that affects the transcriptional or translational regulation of a coding sequence operably linked thereto, wherein the element is not normally found in nature associated or operatively linked to the coding sequence. Heterologous regulatory elements can be promoters, enhancer regions, transcriptional initiation sites, transcriptional termination signals (e.g., poly adenylation signals), translational initiation sequences, etc. Promoters can be constitutive promoters (e.g., those derived from housekeeping genes whose transcription rate is relatively constant, or some viral promoters), inducible promoters (e.g., the metallothionin promoter that is induced in the presence of heavy metals), tissue or cell type specific promoters (e.g., the globin promoters) or promoters derived from animal viruses (e.g., those from CMV, SV40, Adenoviral, Herpesvirus, RSV, HIV, etc.). Enhancers typically increase the level of transcription from operatively linked genes. Enhancers can also be constitutive, tissue specific, and/or inducible (e.g., the CMV enhancer, the SV40 enhancer, the HIV TAR enhancer).

Host cells for use in the invention are eukaryotic host cells, and preferably mammalian cells. Preferably, the cells are also genetically engineered to express a gene of interest. Even more preferably, the host cells are mammalian production cells adapted to grow in cell culture. Examples of such cells commonly used in the industry are CHO, VERO, BHK, HeLa, CV1 (including Cos), MDCK, 293, 3T3, myeloma cell lines (especially murine), PC12 and WI38 cells.

Especially useful host cells are Chinese hamster ovary (CHO) cells, which are widely used for the production of several complex recombinant proteins, e.g. cytokines, clotting factors, and antibodies (Brasel et al., 1996, Blood 88:2004-2012; Kaufman et al., 1988, J.Biol. Chem. 263: 6352-6362; McKinnon et al, 1991, J Mol Endocrinol 6:231-239; Wood et al., 1990, J. Immunol 145:3011-3016). The dihydrofolate reductase (DHFR)-deficient mutant cell line (Urlaub et al., 1980, Proc Natl Acad Sci USA 77:4216-4220), DXB11 and DG-44, are the CHO host cell lines of choice because the efficient DHFR selectable and amplifiable gene expression system allows high level recombinant protein expression in these cells (Kaufman R. J., 1990, Meth Enzymol 185:527-566). In addition, these cells are easy to manipulate as adherent or suspension cultures and exhibit relatively good genetic stability. CHO cells and recombinant proteins expressed in them have been extensively characterized and have been approved for use in clinical manufacturing by regulatory agencies. Other useful host cells, especially for transient transfections, are CV1 (including Cos) and 293 cells. In addition, the invention contemplates genetically engineering hybridoma cell lines to activate the NF-kappa-B transcription factor complex, thereby increasing the production of monoclonal antibodies by the hybridoma cell lines.

For purposes of the invention, a gene for a protein of interest is a gene that encodes a protein of pharmaceutical, medicinal, nutritional, and/or industrial value. Particularly preferred proteins of interest are protein-based drugs. Preferably, the proteins of interest are expressed as extracellular products. Proteins of interest that can be produced using the cell culturing methods and compositions of the invention include but are not limited to a Flt3 ligand, a CD40 ligand, erythropoeitin, thrombopoeitin, calcitonin, Fas ligand, ligand for receptor activator of NF-kappa B (RANKL), TNF-related apoptosis-inducing ligand (TRAIL), ORK/Tek, thymic stroma-derived lymphopoietin, granulocyte colony stimulating factor, granulocyte-macrophage colony stimulating factor, mast cell growth factor, stem cell growth factor, epidermal growth factor, RANTES, growth hormone, insulin, insulinotropin, insulin-like growth factors, parathyroid hormone, interferons, nerve growth factors, glucagon, interleukins 1 through 18, colony stimulating factors, lymphotoxin-β, tumor necrosis factor, leukemia inhibitory factor, oncostatin-M, and various ligands for cell surface molecules Elk and Hek (such as the ligands for eph-related kinases, or LERKS). Descriptions of proteins that can be expressed according to the inventive methods may be found in, for example, Human Cytokines: Handbook for Basic and Clinical Research, Vol. II (Aggarwal and Gutterman, eds. Blackwell Sciences, Cambridge Mass., 1998); Growth Factors:A Practical Approach (McKay and Leigh, eds., Oxford University Press Inc., New York, 1993) and The Cytokine Handbook (A W Thompson, ed.; Academic Press, San Diego Calif.; 1991).

Receptors for any of the aforementioned proteins can also be expressed using the inventive methods and compositions, including both forms of tumor necrosis factor receptor (referred to as p55 and p75), Interleukin-1 receptors (type 1 and 2), Interleukin4 receptor, Interleukin-15 receptor, Interleukin-17 receptor, Interleukin- 18 receptor, granulocyte-macrophage colony stimulating factor receptor, granulocyte colony stimulating factor receptor, receptors for oncostatin-M and leukemia inhibitory factor, receptor activator of NF-kappa B (RANK), receptors for TRAIL (including TRAIL receptors 1, 2, 3, and 4), and receptors that comprise death domains, such as Fas or Apoptosis-Inducing Receptor (AIR).

Other proteins that can be expressed using the inventive methods and compositions include cluster of differentiation antigens (referred to as CD proteins), for example, those disclosed in Leukocyte Typing VI (Proceedings of the VIth International Workshop and Conference; Kishimoto, Kikutani et al., eds.; Kobe, Japan, 1996), or CD molecules disclosed in subsequent workshops. Examples of such molecules include CD27, CD30, CD39, CD40; and ligands thereto (CD27 ligand, CD30 ligand and CD40 ligand). Several of these are members of the TNF receptor family, which also includes 41BB and OX40; the ligands are often members of the TNF family (as are 41BB ligand and OX40 ligand); accordingly, members of the TNF and TNFR families can also be expressed using the present invention.

Proteins that are enzymatically active can also be expressed according to the instant invention. Examples include metalloproteinase-disintegrin family members, various kinases, glucocerebrosidase, superoxide dismutase, tissue plasminogen activator, Factor VIII, Factor IX, apolipoprotein E, apolipoprotein A-I, globins, an IL-2 antagonist, alpha-1 antitrypsin, TNF-alpha Converting Enzyme, and numerous other enzymes. Ligands for enzymatically active proteins can also be expressed by applying the instant invention.

The inventive compositions and methods are also useful for preparation of other types of recombinant proteins, including immunoglobulin molecules or portions thereof, and chimeric antibodies (e.g., an antibody having a human constant region coupled to a murine antigen binding region) or fragments thereof. Numerous techniques are known by which DNA encoding immunoglobulin molecules can be manipulated to yield DNAs capable of encoding recombinant proteins such as single chain antibodies, antibodies with enhanced affinity, or other antibody-based polypeptides (see, for example, Larrick et al., 1989, Biotechnology 7:934-938; Reichmann et al., 1988, Nature 332:323-327; Roberts et al., 1987, Nature 328:731-734; Verhoeyen et al., 1988, Science 239:1534-1536; Chaudhary et al., 1989, Nature 339:394-397). Preparations of fully human antibodies (such as are prepared using transgenic animals, and optionally further modified in vitro), as well as humanized antibodies, can also be used in the invention. The term humanized antibody also encompasses single chain antibodies. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1.

Examples of antibodies or antibody/cytotoxin or antibody/luminophore conjugates contemplated by the invention include those that recognize any one or combination of the above-described proteins and/or the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, IL-1α, IL-1β, IL-4, IL-5, IL-8, IL-10, IL-2 receptor, IL-4 receptor, IL-6 receptor, IL-13 receptor, IL-18 receptor subunits, PDGF-β, VEGF, TGF, TGF-β2, TGF-β1, EGF receptor, VEGF receptor, C5 complement, IgE, tumor antigen CA125, tumor antigen MUC1, PEM antigen, LCG (which is a gene product that is expressed in association with lung cancer), HER-2, a tumor-associated glycoprotein TAG-72, the SK-1 antigen, tumor-associated epitopes that are present in elevated levels in the sera of patients with colon and/or pancreatic cancer, cancer-associated epitopes or proteins expressed on breast, colon, squamous cell, prostate, pancreatic, lung, and/or kidney cancer cells and/or on melanoma, glioma, or neuroblastoma cells, the necrotic core of a tumor, integrin alpha 4 beta 7, the integrin VLA-4, B2 integrins, TRAIL receptors 1, 2, 3, and 4, RANK, RANK ligand, TNF-α, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2 (which is an inhibitor of factor VIIa-tissue factor), MHC I, carcinoembryonic antigen (CEA), alpha-fetoprotein (AFP), tumor necrosis factor (TNF), CTLA-4 (which is a cytotoxic T lymphocyte-associated antigen), Fc-γ-1 receptor, HLA-DR 10 beta, HLA-DR antigen, L-selectin, IFN-γ, Respiratory Syncitial Virus, human imillimolarunodeficiency virus (HIV), hepatitis B virus (HBV), Streptococcus mutans, and Staphlycoccus aureus.

Various fusion proteins can also be expressed using the inventive methods and compositions. Examples of such fusion proteins include proteins expressed as fusion with a portion of an immunoglobulin molecule, proteins expressed as fusion proteins with a zipper moiety, and novel polyfunctional proteins such as a fusion proteins of a cytokine and a growth factor (i.e., GM-CSF and IL-3, MGF and IL-3). WO 93/08207 and WO 96/40918 describe the preparation of various soluble oligomeric forms of a molecule referred to as CD40L, including an immunoglobulin fusion protein and a zipper fusion protein, respectively; the techniques discussed therein are applicable to other proteins.

However, for purposes of this application, the definition of a gene for a protein of interest excludes genes encoding proteins that are typically used as selectable markers in cell culture such as auxotrophic, antimetabolite and/or antibiotic markers. Nevertheless, the invention does include the use of a selectable marker as an aid in selecting cells and/or amplifying clones that are genetically engineered to express a gene of interest and/or a NF-kappa-B gene. Preferably, the selectable marker gene is positioned adjacent to the gene of interest and/or a NF-kappa-B gene such that selection and/or amplification of the marker gene will select and/or amplify the adjacent gene.

Specific examples of genes that encode selectable markers are those that encode antimetabolite resistance such as the DHFR protein, which confers resistance to methotrexate (Wigler et al., 1980, Natl. Acad. Sci. USA 77:3567; O'Hare et al., 1981, Proc. Natl. Acad. Sci. USA 78:1527); the GPT protein, which confers resistance to mycophenolic acid (Mulligan & Berg, 1981, Proc. Natl. Acad. Sci. USA 78:2072), the neomycin resistance marker, which confers resistance to the aminoglycoside G-418 (Colberre-Garapin et al., 1981, J. Mol. Biol. 150:1); the Hygro protein, which confers resistance to hygromycin (Santerre et al., 1984, Gene 30:147); and the Zeocin™ resistance marker (available commercially from Invitrogen). In addition, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively.

Various tissue culture media, including serum-free and/or defined culture media, are commercially available. Tissue culture media is defined, for purposes of the invention, as a media suitable for growth of animal cells, and preferably mammalian cells, in in vitro cell culture. Typically, tissue culture media contains a buffer, salts, energy source, amino acids, vitamins and trace essential elements. Any media capable of supporting growth of the appropriate eukaryotic cell in culture can be used; the invention is broadly applicable to eukaryotic cells in culture, particularly mammalian cells, and the choice of media is not crucial to the invention. Tissue culture media suitable for use in the invention are commercially available from, e.g. ATCC (Manassas, Va.). For example, any one or combination of the following media can be used: RPMI-1640 Medium, Dulbecco's Modified Eagle's Medium, Minimum Essential Medium Eagle, F-12K Medium, Iscove's Modified Dulbecco's Medium. When defined medium that is serum-free and/or peptone-free is used, the medium is usually highly enriched for amino acids and trace elements (see, for example, U.S. Pat. No. 5,122,469 to Mather et al., and U.S. Pat. No. 5,633,162 to Keen et al.).

In the methods and compositions of the invention, cells can be grown in serum-free, protein-free, growth factor-free, and/or peptone-free media. The term “serum-free” as applied to media includes any mammalian cell culture medium that does not contain serum, such as fetal bovine serum. The term “insulin-free” as applied to media includes any medium to which no exogenous insulin has been added. By exogenous is meant, in this context, other than that produced by the culturing of the cells themselves. The term “IGF-1-free” as applied to media includes any medium to which no exogenous Insulin-like growth factor-1 (IGF-1) or analog (such as, for example, LongR³-IGF-1, see below) has been added. The term “growth-factor free” as applied to media includes any medium to which no exogenous growth factor (e.g., insulin, IGF-1) has been added. The term “protein-free” as applied to media includes medium free from exogenously added protein, such as, for example, transferrin and the protein growth factors IGF-1 and insulin. Protein-free media may or may not have peptones. The term “peptone-free” as applied to media includes any medium to which no exogenous protein hydrosylsates have been added such as, for example, animal and/or plant protein hydrosylates. Peptone-free media has the advantages of lower lot to lot variability and fewer filtration problems than media containing plant or animal hydrolysates. Chemically defined media are media in which every component is defined and obtained from a pure source, preferably a non-animal source.

The term “transiently transfected” refers to cells that contain a transfected expression construct, but have not been selected for stable integration into their genome of the expression construct. Thus, “stably transfected” cells are cells that have been selected for stable integration of the expression construct.

The following example is offered by way of illustration, and not by way of limitation.

### EXAMPLE 1

### Co-Expression of NF-kappa-B and Genes of Interest In 293, COS1, and CV1 Cells

The effect of overexpressing NF-kappa-B on expression of a gene of interest, CD40, was examined in 293, COS1 and CV1 cells. The three different host cells were transiently transfected with two plasmids: (1) either an empty vector control (pRC-CMV, available from Invitrogen) or the same vector encoding a uncleavable, caspase-resistant p65/NF-kappa-B subunit; and (2) expression vector pDC409 (described in U.S. Pat. No. 5,763,223) encoding for a gene of interest. Genes of interest used were a CD40Fc fusion gene, and a SEAP gene. The caspase-resistant p65/NF-kappa-B subunit was provided by E. Raines (University of Washington), and is described in Levkau et al., 1999, Nat. Cell. Biol. 1(4):227-33.

Transfections were performed using DEAE-dextran mediated transfection as described (Maniatis et al., 1989, Molecular Cloning: A Laboratory Manual, Book 3; 16.41). Generally, cells were plated out one or two days prior to the transfection. After transfection, growth medium appropriate to the cells was added, and the cells further incubated at 37 degrees C. for 7 days.

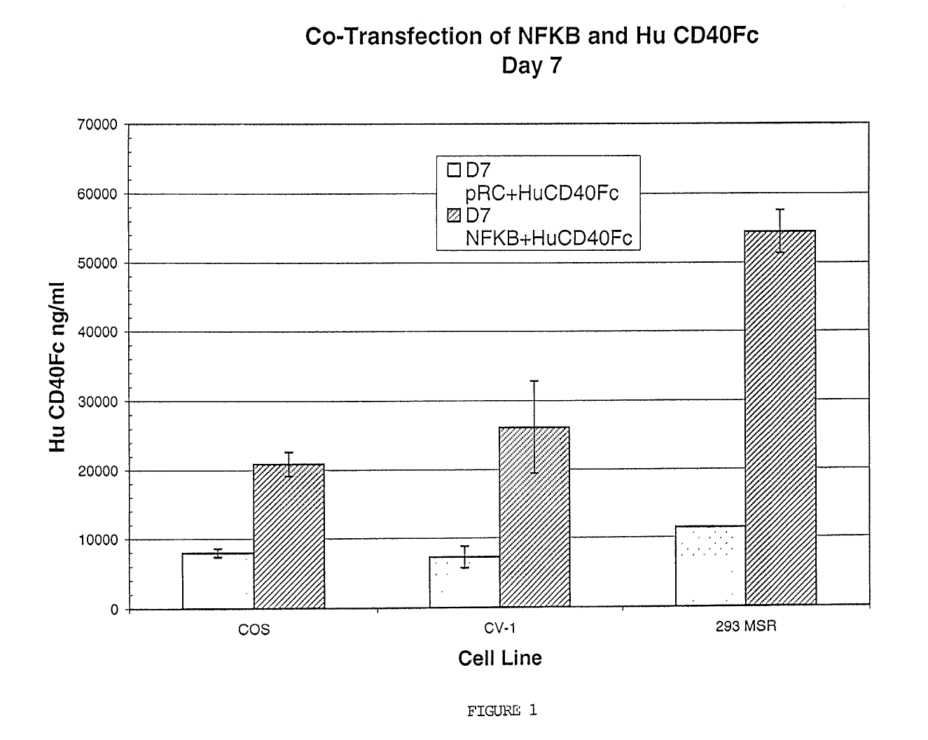

Data on the production of CD40Fc is illustrated in FIG. 1. For all cell types tested, co-expression of the NF-kappa-B subunit dramatically increased production of CD40Fc. Four-fold increases in expression were observed. The results also showed similar increases in production of SEAP as the gene of interest when co-expressed with the NF-kappa-B subunit. Further analysis demonstrated that the increased levels of production of these genes of interest were accompanied by an increase in the levels of the encoding mRNA. The technique was generally applicable to different cell types and different secreted gene products.

The present invention is not to be limited in scope by the specific embodiment described herein, which is intended as a single illustration of aspects of the invention, and functionally equivalent methods and components are within the scope of the invention. Indeed, various modifications of the invention, in addition to those shown and described herein will become apparent to those skilled in the art from the foregoing description and accompanying drawing, and especially in light of the teachings of the various references cited herein, the disclosures of which are incorporated by reference in their entirety. Such modifications are intended to fall within the scope of the appended claims.

What is claimed is:

1. An eukaryotic host cell genetically engineered to activate the NF-kappa-B transcription factor complex, and to express a protein of interest as an extracellular product.

2. The host cell of claim 1 wherein the host cell is genetically engineered to express an NF-kappa-B transcription factor.

3. The host cell of claim 2 wherein the NF-kappa-B transcription factor is selected from the group consisting of p65, p50, cRel, p52 and RelB.

4. The host cell of claim 2 wherein the NF-kappa-B transcription factor is expressed under control of a heterologous regulatory element.

5. The host cell of claim 4, wherein the heterologous regulatory element is a viral promoter.

6. The host cell of claim 5, wherein the viral promoter is selected from the group consisting of a CMV promoter, an SV40 promoter, an RSV promoter and an adenoviral promoter.

7. The host cell of claim 1, wherein the protein of interest is selected from the group consisting of a soluble TNF receptor, a soluble IL-4 receptor, a soluble IL-1 type II receptor, a soluble Flt3 ligand, a soluble CD40 ligand, CD39, CD30, CD27, a TEK/Ork, IL-15, a soluble IL-15 receptor, Ox 40, GM-CSF, RANKL, RANK, TRAIL, a soluble TRAIL receptor, tissue plasminogen activator, Factor VIII, Factor IX, apolipoprotein E, apolipoprotein A-I, an IL-2 receptor, an IL-2 antagonist, alpha-1 antitrypsin, calcitonin, growth hormone, insulin, insulinotropin, insulin-like growth factors, parathyroid hormone, interferons, superoxide dismutase, glucagon, an erythropoeitin, an antibody, glucocerebrosidase, an Fc-fusion protein, globins, nerve growth factors, interleukins, colony stimulating factors, and a cytokine.

8. The host cell of claim 1, wherein the host cell is further genetically engineered to express a selectable marker.

9. The host cell of claim 1, wherein the host cell is a mammalian cell.

10. The host cell of claim 9, wherein the host cell is selected from the group consisting of CHO, VERO, BHK, HeLa, CV1, COS, MDCK, 293, 3T3, myeloma, PC12 and WI38.

11. The host cell of claim 1, wherein the host cell is adapted to grow in protein-free medium.

12. The host cell of claim 2, wherein the NF-kappa-B transcription factor is a caspase resistant p65 mutant.

13. The host cell of claim 2, wherein the host cell is genetically engineered to express a second NF-kappa-B transcription factor.

14. A method of producing a protein of interest, the method comprising culturing an eukaryotic host cell genetically engineered to activate the NF-kappa-B transcription factor complex, and to express a protein of interest as an extracellular product, under conditions such that the protein of interest is expressed and secreted.

15. The method of claim 14, further comprising collecting the protein of interest.

16. The method of claim 14, wherein the host cell is genetically engineered to express an NF-kappa-B transcription factor.

17. The method of claim 16, wherein the NF-kappa-B transcription factor is selected from the group consisting of p65, p50, cRel, p52 and RelB.

18. The method of claim 16, wherein the NF-kappa-B transcription factor is expressed under control of a heterologous regulatory element.

19. The method of claim 18, wherein the heterologous regulatory element is a viral promoter.

20. The method of claim 19, wherein the viral promoter is selected from the group consisting of a CMV promoter, an SV40 promoter, an RSV promoter and an adenoviral promoter.

21. The method of claim 14, wherein the protein of interest is selected from the group consisting of a soluble TNF receptor, a soluble IL-4 receptor, a soluble IL-1 type II receptor, a soluble Flt3 ligand, a soluble CD40 ligand, CD39, CD30, CD27, a TEK/Ork, IL-15, a soluble IL-15 receptor, Ox 40, GM-CSF, RANKL, RANK, TRAIL, a soluble TRAIL receptor, tissue plasminogen activator, Factor VIII, Factor IX, apolipoprotein E, apolipoprotein A-I, an IL-2 receptor, an IL-2 antagonist, alpha-1 antitrypsin, calcitonin, growth hormone, insulin, insulinotropin, insulin-like growth factors, parathyroid hormone, interferons, superoxide dismutase, glucagon, an erythropoeitin, an antibody, glucocerebrosidase, an Fc-fusion protein, globins, nerve growth factors, interleukins, colony stimulating factors, and a cytokine.

22. The method of claim 14, wherein the host cell is further genetically engineered to express a selectable marker.

23. The method of claim 14, wherein the host cell is a mammalian cell.

24. The method of claim 23, wherein the host cell is selected from the group consisting of CHO, VERO, BHK, HeLa, CV1, MDCK, 293, 3T3, myeloma, PC12 and WI38.

25. The method of claim 14, wherein the host cell is cultured in protein-free medium.

26. The method of claim 15, wherein the NF-kappa-B transcription factor is a caspase resistant p65 mutant.

27. The method of claim 14, wherein the host cell is transiently transfected.

28. The method of claim 14, wherein the host cell is stably transformed.

29. The method of claim 15, wherein the host cell is genetically engineered to express a second NF-kappa-B transcription factor.

30. A method of producing an eukaryotic cell for production of a protein of interest, the method comprising genetically engineering an eukaryotic cell to express a gene that encodes a protein of interest as an extracellular product, and to activate the NF-kappa-B transcription factor complex.

31. A method of producing a mammalian cell line capable of growth in protein-free medium, the method comprising exposing cells that have been genetically engineered to activate the NF-kappa-B transcription factor complex to protein-free medium, and isolating a cell line that grows in protein-free medium.

32. The method of claim 31, further comprising exposing the cells to peptone-free medium, and isolating a cell line that grows in peptone-free medium.

33. A method of producing an eukaryotic cell for production of a protein of interest, the method comprising genetically engineering an eukaryotic cell to express a protein of interest as an extracellular product, wherein the eukaryotic cell has been genetically engineered to activate the NF-kappa-B transcription factor complex.

34. A method of producing an eukaryotic cell for production of a protein of interest, the method comprising genetically engineering a cell to activate the NF-kappa-B transcription factor complex, wherein the eukaryotic cell expresses a protein of interest as an extracellular product.
